(19) United States
(12) Patent Application Publication
(10) Pub. No.: US 2012/0073472 A1
(43) Pub. Date: Mar. 29, 2012

(54) REACTIVE DERIVATIVES ON THE BASIS OF DIANHYDROHEXITOL-BASED ISOCYANATES

(75) Inventors: Emmanouil Spyrou, Schermbeck (DE); Jan Pfeffer, Essen (DE); Holger Loesch, Herne (DE); Marion Ebbing-Ewald, Marl (DE); Heinz Grosse-Beck, Bottrop (DE)

(73) Assignee: EVONIK DEGUSSA GMBH, Essen (DE)

(21) Appl. No.: 13/376,780

(22) PCT Filed: Apr. 28, 2010
(86) PCT No.: PCT/EP2010/055700

§ 371 Date: Dec. 7, 2011

(30) Foreign Application Priority Data
Jul. 1, 2009 (DE) 102009027395.6

(51) Int. Cl. C09D 7/12; C07C 267/00; C07D 251/34; C07C 237/00; C07D 493/04; C07D 229/00

(52) U.S. Cl. 106/287.21; 106/287.2; 549/464; 548/951; 544/192; 564/198; 564/252

(57) ABSTRACT

The invention relates to reactive derivatives on the basis of dianhydrohexitol-based isocyanates.

Isocyanates, as valuable building blocks for polyurethane chemistry, have already long been known and described. Thus, aromatic isocyanates such as methanediphenyl diisocyanate (MDI) and tolyl diisocyanate (TDI), for example, have been used in many 100 000s of t for decades, for polyurethane foams, for example.

Aliphatic isocyanates, such as hexamethylene diisocyanate (HDI) or isophorone diisocyanate (IPDI), for example, were commercialized later. As a result of their particular weather-stable properties they find their use, for example, in UV-resistant automobile finishes.

Appearing even later on the timeline are isocyanates formed from renewable raw materials. Lysine diisocyanate is suitable, for example, particularly for medical uses, since derivatives of such nature-similar substances have proven biocompatible.

Bicyclic isocyanates, such as norbornane diisocyanate, for example, lead to derivatives with high Tg, owing to the rigid structure, and are therefore used primarily for powder coatings.

The continual development of new isocyanates illustrates the demand for these reactive products with a greater breadth of variation in properties.

Isocyanates formed from renewable raw materials are playing an ever greater part not least, quite simply, for reasons of sustainability and also for reasons of cost.

This explains, inter alia, the development of isocyanates based on renewable and inexpensive sugars, such as 1:4-3:6 dianhydrohexitols for example (J. Thiem et al., Macromol. Chem. Phys. 202, 3410-3419, 2001). This literature reference describes the preparation of diisocyanates from the corresponding diamines and the subsequent reaction with certain monomeric alcohols, amines, and thiols.

Used as isocyanate component in this context were 2,5-diisocyanato-1,4:3,6-dianhydro-2,5-dideoxy-D-mannitol (I), 2,5-diisocyanato-1,4:3,6-dianhydro-2,5-dideoxy-D-glucitol (II) and 2,5-diisocyanato-1,4:3,6-dianhydro-2,5-dideoxy-L-iditol (III), with the formulae

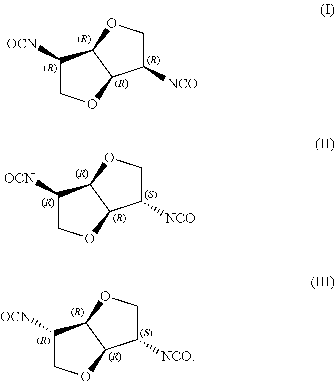

In the text below, these and also the isomers not depicted are called, for simplification, dianhydrohexitol-based diisocyanates.

Although monomeric diisocyanates of this kind, formed from renewable raw materials, with heterocyclic bicyclic rings, meet the demand for specific isocyanate building blocks having particular properties, they have the disadvantage of offering a limited selection of reactive structures for applications in the coatings, adhesives, sealing, and plastics sector. Moreover, monomeric diisocyanates are generally toxic, often sensitizing too, and must therefore usually be labeled with a T (toxic) at a level of >2% by weight. Above 20% by weight of monomers, the R phrases R36, R37, R38 are added as well (irritant to the eyes, respiratory organs, and skin).

Isocyanates based on dianhydrohexitols possess two fused heterocyclic rings, which at relatively high temperatures and/or with specific catalysts tend toward polymerization and decomposition phenomena. Here, presumably, there is a ring-opening polymerization of the heterocycles. Moreover, the steric environment of dianhydrohexitol-based diisocyanates is greatly hindered, and so the reactivity may be very different depending on isomer. For the reasons stated, the possibility for converting dianhydrohexitol-based diisocyanates into reactive derivatives is questionable and, consequently, neither published nor known.

It was an object of this invention to provide reactive isocyanate components which on the one hand are based on renewable raw materials and on the other hand have heterocyclic basic structures, but nevertheless have a low monomer content. The intention, moreover, was that the known parent structure of the dianhydrohexitols should be transferred to further structures preferred in isocyanate chemistry.

The object according to the invention has been solved by preparation of low-monomer-content derivatives based on dianhydrohexitol-based diisocyanates. Surprisingly it has been found that dianhydrohexitol-based diisocyanates can be converted by appropriate processes and reagents into dimers, trimers, NCO prepolymers, blocked diisocyanates, allophanates and carbodiimides.

Subject matter of the invention are derivates of dianhydrohexitol-based diisocyanates, the derivatives possessing free and/or blocked NCO groups and the monomeric diisocyanates content being less than 20% by weight, preferably less than 2% by weight, selected from

1) dimers (uretdiones),
2) trimers (isocyanurates),
3) NCO prepolymers having free or blocked NCO groups,
4) blocked diisocyanates,
5) allophanates,
6) carbodiimides and/or uretonimines;
alone or in mixtures.

Preferred subject matter of the invention are derivates of dianhydrohexitol-based diisocyanates Ito III as starting compounds:

2,5-diisocyanato-1,4:3,6-dianhydro-2,5-dideoxy-D-mannitol (I), 2,5-diisocyanato-1,4:3,6-dianhydro-2,5-dideoxy-D-glucitol (II) and 2,5-diisocyanato-1,4:3,6-dianhydro-2,5-dideoxy-L-iditol (III), with the formulae

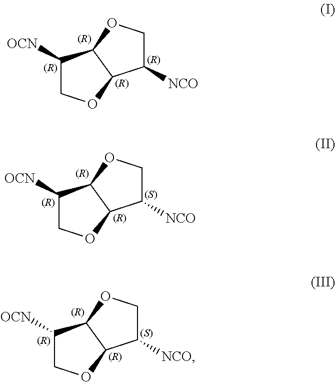

the derivatives possessing free and/or blocked NCO groups and the monomeric diisocyanates content being less than 20% by weight,
selected from
1) dimers (uretdiones),
2) trimers (isocyanurates),
3) NCO prepolymers having free or blocked NCO groups,
4) blocked diisocyanates,
5) allophanates,
6) carbodiimides and/or uretonimines;
alone or in mixtures.

Also subject matter of the invention are processes for preparing these derivatives, and also their use for producing coating, adhesive, sealant or plastics products.

1) Subject matter of the invention are dimers (uretdiones) based on dianhydrohexitol-based diisocyanates, preferably of the formula I-III. The conversion of nonheterocyclic diisocyanates into uretdiones has been known for a long time and is described in U.S. Pat. No. 4,476,054, U.S. Pat. No. 4,912,210, U.S. Pat. No. 4,929,724, and EP 0 417 603, for example. A comprehensive overview of industrially relevant processes for the dimerization of isocyanates to uretdiones is supplied by J. Prakt. Chem. 336 (1994) 185-200. In general the reaction of isocyanates to uretdiones takes place in the presence of soluble dimerization catalysts, such as dialkylaminopyridines, trialkylphosphines, phosphoramides or imidazoles, for example. The reaction—carried out optionally in solvents, but preferably in the absence of solvents—is stopped by addition of catalyst poisons when a desired conversion has been reached. Excess monomeric isocyanate is removed subsequently by short-path evaporation. If the catalyst is sufficiently volatile, the reaction mixture can be freed from the catalyst in the course of the removal of monomer. In that case there is no need to add catalyst poisons. A broad range of isocyanates are suitable in principle for preparing polyisocyanates containing uretdione groups. Particularly suitable are, however, generally diisocyanates which contain at least one aliphatic NCO group, such as hexamethylene diisocyanate (HDI) or isophorone diisocyanate (IPDI), for example. Diisocyanates which contain only cycloaliphatic NCO groups are particularly difficult to convert to uretdiones. Such conversion was for many decades considered impossible, until for the first time, in WO 2004/005364, (pp. 15-19), a description was given of the preparation of suitable catalysts and the resulting isocyanates, containing uretdione groups, on the basis of a purely cycloaliphatic diisocyanate (diisocyanatodicyclohexylmethane (H₁₂MDI)). The catalyst used in that case was Na triazolate. Using the same catalyst in the case of dianhydrohexitol-based diisocyanates, which also contain only cycloaliphatic isocyanate groups, results, following development of foam and heat, to insoluble polymers without detectable free NCO groups. This underlines the reactive and unpredictable nature of dianhydrohexitol-based diisocyanates.

The preparation of dimers (uretdiones) based on dianhydrohexitol-based diisocyanates is finally accomplished, however, in dichloromethane at room temperature using, for example, 4-(dimethylamino)pyridine as catalyst. This results in soluble products which (according to 13-C-NMR) contain a considerable proportion of uretdione groups. The free NCO content is between 1%-42% by weight, the uretdione content between 1% and 42% by weight, and the monomer content between 0.5% and 98% by weight. This product can be separated largely to completely from excess monomer content by means of a suitable gentle distillation method (e.g. short-path distillation, thin-film distillation, bulb-tube distillation). The monomer content after this distillation is 0%-20% by weight, preferably 0.1%-2% by weight.

The further conversion of these polyisocyanates containing uretdione groups (dimers) to form hardeners containing uretdione groups incorporates the reaction of the free NCO groups with hydroxyl-containing monomers or polymers, such as, for example, polyesters, polythioethers, polyethers, polycaprolactams, polyepoxides, polyesteramides, polyurethanes or low molecular weight di-, tri- and/or tetraalcohols as chain extenders and optionally monoamines and/or monoalcohols as chain terminators, and has already been frequently described (EP 0 669 353, EP 0 669 354, DE 30 30 572, EP 0 639 598 or EP 0 803 524). Preferred hardeners containing uretdione groups have a free NCO content of less than 5% by weight and a uretdione groups content of 2% to 25% by weight (calculated as C₂N₂O₂, molecular weight 84). Polyesters and monomeric dialcohols are preferred. Besides the uretdione groups, the hardeners may also contain isocyanurate, biuret, allophanate, urethane and/or urea structures.

2) The subject matter of the invention are trimers (isocyanurates) based on dianhydrohexitol-based diisocyanates, preferably of the formula I-III. In principle, isocyanurates are obtained by catalytic trimerization of suitable isocyanates. Suitable isocyanates are, for example, aromatic, cycloaliphatic and aliphatic polyisocyanates having a functionality of two or more. Catalysts contemplated include, for example, tertiary amines (U.S. Pat. No. 3,996,223), alkali metal salts of carboxylic acids (CA 2 113 890; EP 056 159), quaternary ammonium salts (EP 798 299; EP 524 501; U.S. Pat. No. 4,186,255; U.S. Pat. No. 5,258,482; U.S. Pat. No. 4,503,226; U.S. Pat. No. 5,221,743), aminosilanes (EP 197 864; U.S. Pat. No. 4,697,014) and quaternary hydroxyalkylammonium salts (EP 017 998; U.S. Pat. No. 4,324,879). Depending on the catalyst, it is also possible to use various co-catalysts, examples being OH-functionalized compounds or Mannich bases formed from secondary amines and aldehydes and/or ketones.

For trimerization, the polyisocyanates can be reacted until the desired conversion is achieved in the presence of the catalyst, optionally with use of solvents and/or auxiliaries. In this context, the term “partial trimerization” is also used, since the target conversion is usually well below 100%. The reaction is thereafter terminated by deactivation of the catalyst. This is done by adding a catalyst inhibitor such as p-toluenesulfonic acid, hydrogen chloride or dibutyl phosphate, for example, and results automatically in a possibly unwanted contamination of the resultant polyisocyanate containing isocyanurate groups. Particularly advantageous in the context of the trimerization of isocyanates on an industrial scale is the use of quaternary hydroxyalkylammonium carboxylates as oligomerization catalysts. This type of catalyst is thermally labile and permits a deliberate thermal deactivation, thereby removing the need to stop the trimerization on attainment of the desired conversion by addition of potentially quality-lowering inhibitors.

Accordingly, dianhydrohexitol-based diisocyanates are also trimerized with quaternary hydroxyalkylammonium carboxylates at temperatures of approximately 40-140° C. The free NCO content after the reaction is 1%-42% by weight, preferably 20%-40% by weight. The monomer content is between 0.5% and 98% by weight, preferably 40%-95% by weight. Here as well, excess diisocyanate can be removed by distillation.

Furthermore, the NCO-containing trimers of the invention can also be blocked with conventional blocking agents such as, for example, phenols such as phenol, and p-chlorophenol, alcohols such as benzyl alcohol, oximes such as acetone oxime, methyl ethyl ketoxime, cyclopentanone oxime, cyclohexanone oxime, methyl isobutyl ketoxime, methyl tert-butyl ketoxime, diisopropyl ketoxime, diisobutyl ketoxime, or acetophenone oxime, N-hydroxy compounds such as N-hydroxysuccinimide or hydroxypyridines, lactams such as ε-caprolactam, CH-acidic compounds such as ethyl acetoacetate or malonic esters, amines such as diisopropylamine, heterocyclic compounds having at least one heteroatom such as mercaptans, piperidines, piperazines, pyrazoles, imidazoles, triazoles and tetrazoles, α-hydroxybenzoic esters such as glycolic esters or hydroxamic esters such as benzyl methacrylohydroxamate, and can be used in thermosetting 1-component formulations.

Particularly suitable blocking agents are acetone oxime, methyl ethyl ketoxime, acetophenone oxime, diisopropylamine, 3,5-dimethylpyrazole, 1,2,4-triazole, ε-caprolactam, butyl glycolate, benzyl methacylohydroxamate or methyl p-hydroxybenzoate.

3) Subject matter of the invention are NCO-containing prepolymers having free and/or blocked NCO groups on the basis of dianhydrohexitol-based diisocyanates, preferably of the formula I-III, and polyols, obtainable by reacting dianhydrohexitol-based diisocyanates and at least one at least difunctional polyol in the NCO/OH ratio of 1.5-2:1 at 20-120° C. The monomer content after the reaction can be between 0.5%-20% by weight.

For the preparation of the NCO-containing prepolymers of the invention, dianhydrohexitol-based diisocyanates, preferably of the formula I-III, optionally in a mixture with other aliphatic or cycloaliphatic diisocyanates, are introduced and an at least difunctional polyol is added. The NCO/OH ratio here is between 1.5:1 and 2:1. In general, the reaction takes place in the presence of a catalyst at 20-120° C. The reaction may be carried out in suitable assemblies, stirred tanks, static mixers, tube reactors, compounders, extruders or other reaction spaces with or without a mixing function. The reaction may take place in solvent or else solventlessly.

Suitable organic solvents contemplated include all liquid substances which do not react with other ingredients, examples being acetone, ethyl acetate, butyl acetate, xylene, Solvesso 100, Solvesso 150, methoxypropyl acetate and dibasic esters.

The monomer content of the prepolymer thus prepared can be lowered further by means of an appropriate distillation, examples being short-path distillation or thin-film distillation. The preferred monomer content after distillation is <2% by weight, more preferably <0.5% by weight.

Examples of diisocyanates suitable for blending with dianhydrohexitol-based diisocyanates are hexamethylene diisocyanate (HDI), isophorone diisocyanate (IPDI), 4,4′-methylenebis(cyclohexyl isocyanate) (H₁₂MDI), 2-methylpentane-methylene 1,5-diisocyanate (MPDI), trimethylhexamethylene 1,6-diisocyanate (TMDI), or m-tetramethylxylylene diisocyanate (TMXDI).

Catalysts suitable for the reaction are available commercially and are based in general on metal compounds or transition metal compounds based on aluminum, tin, zinc, titanium, manganese, bismuth, or zirconium, such as dibutyltin dilaurate, bismuth neodecanoate, zinc octoate, titanium tetrabutoxide or zirconium octoate, for example, or else on tertiary amines such as 1,4-diazabicyclo[2.2.2]octane, for example.

Examples of polyols used are ethylene glycol, 1,2-, 1,3-propanediol, diethylene, dipropylene, triethylene, and tetraethylene glycol, 1,2-, 1,4-butanediol, 1,3-butylethylpropanediol, 1,3-methylpropanediol, 1,5-pentanediol, bis(1,4-hydroxymethyl)cyclohexane (cyclohexanedimethanol), glycerol, hexanediol, neopentylglycol, trimethylolethane, trimethylolpropane, pentaerythritol, bisphenol A, B, C, F, norbornylene glycol, 1,4-benzyldimethanol, -ethanol, 2,4-dimethyl-2-ethylhexane-1,3-diol, 1,4- and 2,3-butylene glycol, di-1′-hydroxyethylbutanediol, 1,5-pentanediol, 1,6-hexanediol, 1,8-octanediol, decanediol, dodecanediol, neopentylglycol, cyclohexanediol, 3(4),8(9)-bis(hydroxymethyl)tricyclo-[5.2.1.0^{2,6}]decane (Dicidol), 2,2-bis(4-hydroxycyclohexyl)propane, 2,2-bis[4-(β-hydroxyethoxy)phenyl]propane, 2-methylpropane-1,3-diol, 2-methylpentane-1,5-diol, 2,2,4(2,4,4)-trimethylhexane-1,6-diol, hexane-1,2,6-triol, butane-1,2,4-triol, tris(β-hydroxyethyl)isocyanurate, mannitol, sorbitol, polypropylene glycols, polybutylene glycols, xylylene glycol or neopentylglycol hydroxypivalate, alone or in mixtures.

Particularly preferred are 1,4-butanediol, 1,2-propanediol, cyclohexanedimethanol, hexanediol, neopentylglycol, decanediol, dodecanediol, trimethylolpropane, ethylene glycol, triethylene glycol, pentane-1,5-diol, hexane-1,6-diol, 3-methylpentane-1,5-diol, neopentylglycol, 2,2,4(2,4,4)-trimethylhexanediol and neopentylglycol hydroxypivalate. They are used alone or in mixtures.

Also suitable as polyols are diols and polyols which contain further functional groups. These are the conventional, linear or branched hydroxyl-containing polyesters, polycarbonates, polycaprolactones, polyethers, polythioethers, polyesteramides, polyacrylates, polyurethanes or polyacetals. They preferably have a number-average molecular weight of 62 to 20 000, more preferably 134-4000. The hydroxyl-containing polymers used are preferably polyesters, polyethers, polyacrylates, polyurethanes, polyvinyl alcohols and/or polycarbonates having an OH number of 5-500 (in mg KOH/gram).

Preference is given to linear or branched hydroxyl-containing polyesters—polyester polyols—or mixtures of such polyesters. They are prepared, for example, by reaction of diols with substoichiometric amounts of dicarboxylic acids, corresponding dicarboxylic anhydrides, corresponding dicarboxylic esters of lower alcohols, lactones, or hydroxycarboxylic acids.

Diols suitable for preparing the preferred polyester polyols, in addition to those diols specified above, include 2-methylpropanediol, 2,2-dimethylpropanediol, diethylene glycol, dodecane-1,12-diol, 1,4-cyclohexanedimethanol and 1,2- and 1,4-cyclohexanediol.

Dicarboxylic acids or derivatives that are suitable for preparing the polyester polyols may be aliphatic, cycloaliphatic, aromatic and/or heteroaromatic in nature and may optionally be substituted, by halogen atoms, for example, and/or unsaturated.

The preferred dicarboxylic acids or derivatives include succinic, adipic, suberic, azelaic, and sebacic acid, 2,2,4(2,4,4)-trimethyladipic acid, phthalic acid, phthalic anhydride, isophthalic acid, terephthalic acid, dimethyl terephthalate, tetrahydrophthalic acid, maleic acid, maleic anhydride and dimeric fatty acids.

Suitable polyester polyols are also those which can be prepared in a known way by ring opening from lactones, such as caprolactone, and simple diols as starter molecules. Monoesters and polyesters formed from lactones as well, such as from ε-caprolactone or hydroxycarboxylic acids, e.g., hydroxypivalic acid, ε-hydroxydecanoic acid, ε-hydroxycaproic acid, thioglycolic acid, can be used as starting materials for preparing the polymers G). Polyesters formed from the polycarboxylic acids stated above (page 6) and/or derivatives thereof and from polyphenols, such as hydroquinone, bisphenol A, 4,4′-dihydroxybiphenyl or bis(4-hydroxyphenyl) sulfone; polyesters of carbonic acid, which are obtainable from hydroquinone, diphenylolpropane, p-xylylene glycol, ethylene glycol, butanediol or hexane-1,6-diol and other polyols by customary condensation reactions, as for example with phosgene or diethyl and/or diphenyl carbonate, or from cyclic carbonates, such as glycol carbonate or vinylidene carbonate, by polymerization in a known way; polyesters of silicic acid, polyesters of phosphoric acid, e.g., from methane, ethane, β-chloroethane, benzene- or styrenephosphoric acid or derivatives thereof, such as phosphoric acid chlorides or phosphoric acid esters, for example, and from polyalcohols or polyphenols of the type specified above; polyesters of boric acid; polysiloxanes, such as the products, for example, obtainable by hydrolysis of dialkyldichlorosilanes with water and subsequent treatment with polyalcohols, the products obtainable by addition reaction of polysiloxane dihydrides with olefins, such as allyl alcohol or acrylic acid, are suitable as starting materials for the preparation of the polyols.

The polyesters can be obtained in a conventional way by condensation in an inert gas atmosphere at temperatures from 100 to 260° C., preferably 130 to 220° C., in the melt or in an azeotropic regime, as is described, for example, in Methoden der Organischen Chemie (Houben-Weyl); volume 14/2, pages 1 to 5, 21 to 23, 40 to 44, Georg Thieme Verlag, Stuttgart, 1963, or in C. R. Martens, Alkyd Resins, pages 51 to 59, Reinhold Plastics Appl. Series, Reinhold Publishing Comp., New York, 1961.

The diols and dicarboxylic acids and/or derivatives thereof that are used for preparing the polyester polyols can be employed in any desired mixtures.

It is also possible to use mixtures of polyester polyols and diols.

Likewise possible for use with preference are (meth)acrylates and poly(meth)acrylates containing OH groups. They are prepared by the copolymerization of (meth)acrylates, with certain components carrying OH groups while others do not. Accordingly, a randomly distributed polymer containing OH groups is produced, that carries none, one or a large number of OH group(s). Polymers of this kind are described in High solids hydroxy acrylics with tightly controlled molecular weight, van Leeuwen, Ben., SC Johnson Polymer, Neth. PPCJ, Polymers Paint Colour Journal (1997), 187(4392), 11-13;

Special techniques for synthesis of high solid resins and applications in surface coatings. Chakrabarti, Suhas; Ray, Somnath. Berger Paints India Ltd., Howrah, India. Paintindia (2003), 53(1), 33-34, 36, 38-40;

VOC protocols and high solid acrylic coatings. Chattopadhyay, Dipak K.; Narayan, Ramanuj; Raju, K. V. S, N. Organic Coatings and Polymers Division, Indian Institute of Chemical Technology, Hyderabad, India. Paintindia (2001), 51(10), 31-42.

Suitable polyols are also the reaction products of polycarboxylic acids and glycidyl compounds, as are described in DE-A 24 10 513, for example.

Examples of glycidyl compounds which can be used are esters of 2,3-epoxy-1-propanol with monobasic acids, having 4 to 18 carbon atoms, such as glycidyl palmitate, glycidyl laurate and glycidyl stearate, alkylene oxides having 4 to 18 carbon atoms, such as butylene oxide, and glycidyl ethers, such as octyl glycidyl ether.

Suitable polyols are also those which as well as an epoxide group also carry at least one further functional group, such as, for example, carboxyl, hydroxyl, mercapto or amino groups, capable of reaction with an isocyanate group. Particularly preferred are 2,3-epoxy-1-propanol and epoxidized soybean oil.

It is possible to use any desired combinations of these compounds.

The prepolymers of the invention may also comprise chain extenders, such as low molecular weight polyhydric alcohols or amino alcohols, for example.

Furthermore, the NCO-containing prepolymers of the invention can also be completely or partially blocked with conventional blocking agents such as, for example, phenols such as phenol, and p-chlorophenol, alcohols such as benzyl alcohol, oximes such as acetone oxime, methyl ethyl ketoxime, cyclopentanone oxime, cyclohexanone oxime, methyl isobutyl ketoxime, methyl tert-butyl ketoxime, diisopropyl ketoxime, diisobutyl ketoxime, or acetophenone oxime, N-hydroxy compounds such as N-hydroxysuccinimide or hydroxypyridines, lactams such as ε-caprolactam, CH-acidic compounds such as ethyl acetoacetate or malonic esters, amines such as diisopropylamine, heterocyclic compounds having at least one heteroatom such as mercaptans, piperidines, piperazines, pyrazoles, imidazoles, triazoles and tetrazoles, α-hydroxybenzoic esters such as glycolic esters or hydroxamic esters such as benzyl methacrylohydroxamate, and can be used in thermosetting 1-component formulations.

Particularly suitable blocking agents are acetone oxime, methyl ethyl ketoxime, acetophenone oxime, diisopropylamine, 3,5-dimethylpyrazole, 1,2,4-triazole, ε-caprolactam, butyl glycolate, benzyl methacylohydroxamate or methyl p-hydroxybenzoate.

4) Subject matter of the invention are also completely or partially blocked diisocyanates, based on dianhydrohexitol-based diisocyanates, preferably of the formula I-III.

The blocking (temporary deactivation) of isocyanates has already been known for a long time. It involves reacting the isocyanate component with what is called a blocking agent, which is stable under storage conditions (typically up to 50° C.) for several weeks or else for at least a year at room temperature. At higher temperatures (upward of 120-180° C.), the blocking agent is eliminated and hence the original reactivity of the NCO groups is re-established. The blocking itself takes place at temperatures between room temperature and 220° C. This reaction may take place solventlessly or else in a solvent, with solvents contemplated including reaction media that are merely inert toward NCO groups. Suitable organic solvents contemplated include, for example, all liquid substances which do not react with other ingredients, examples being acetone, ethyl acetate, butyl acetate, xylene, Solvesso 100, Solvesso 150, methoxypropyl acetate and dibasic esters. Suitable and particularly preferred blocking agents are identical to those specified above under 3).

The blocking reaction can be carried out in suitable assemblies, stirred tanks, static mixers, tube reactors, compounders, extruders or other reaction spaces with or without a mixing function. The reaction is carried out at temperatures between room temperature and 220° C., preferably between 40° C. and 120° C., and lasts, depending on temperature and reaction components, for between a few seconds and several hours. A reaction time between 30 minutes and 24 hours is preferred. The ratio between NCO component and blocking agent is NCO/blocking agent=1:1 to 1:1.2, preferably 1:1 to 1:1.05. The end product does not possess any notable free NCO groups (NCO content<0.5% by weight).

5) Subject matter of the invention are allophanates based on dianhydrohexitol-based diisocyanates, preferably of the formula I-III. Allophanates are reaction products of urethanes and (poly-)isocyanates, also from 2). They can alternatively be formed also through the addition reaction of alcohols with uretdiones, such as 1). Alcohols are subjected to addition reaction in substoichiometric amount, in a known urethane reaction, with dianhydrohexitol-based diisocyanates, and, following complete reaction, (according to NCO content analysis), an allophanatization catalyst (e.g. zinc octoate) is added and the allophanatization reaction proper is carried out at a relatively high temperature (generally 80-140° C.) over a prolonged time (generally 30 minutes to 8 hours), until change in the NCO content is no longer detected. The excess of free monomer can be removed by means of a suitable distillation (e.g. short-path distillation or thin-film distillation). The preferred monomer content after distillation is <2% by weight, more preferably <0.5% by weight.

Alcohols contemplated include, in particular, mono- and polyfunctional monomeric alcohols, examples being methanol, ethanol, propanol and isomers, butanol and isomers, pentanol and isomers, hexanol and isomers, octanol and isomers, decanol and isomers, dodecanol and isomers, ethylene glycol, 1,2-, 1,3-propanediol, diethylene, dipropylene, triethylene, and tetraethylene glycol, 1,2-, 1,4-butanediol, 1,3-butylethylpropanediol, 1,3-methylpropanediol, 1,5-pentanediol, bis(1,4-hydroxymethyl)cyclohexane (cyclohexanedimethanol), glycerol, hexanediol, neopentylglycol, trimethylolethane, trimethylolpropane, pentaerythritol, bisphenol A, B, C, F, norbornylene glycol, 1,4-benzyldimethanol, -ethanol, 2,4-dimethyl-2-ethylhexane-1,3-diol, 1,4- and 2,3-butylene glycol, di-β-hydroxyethylbutanediol, 1,5-pentanediol, 1,6-hexanediol, 1,8-octanediol, decanediol, dodecanediol, neopentylglycol, cyclohexanediol, 3(4),8(9)-bis(hydroxymethyl)tricyclo-[5.2.1.0^{2,6}]decane (Dicidol), 2,2-bis(4-hydroxycyclohexyl)propane, 2,2-bis[4-(β-hydroxyethoxy)phenyl]propane, 2-methylpropane-1,3-diol, 2-methylpentane-1,5-diol, 2,2,4(2,4,4)-trimethylhexane-1,6-diol, hexane-1,2,6-triol, butane-1,2,4-triol, tris(β-hydroxyethyl)isocyanurate, mannitol, sorbitol, polypropylene glycols, polybutylene glycols, xylylene glycol or neopentylglycol hydroxypivalate, hydroxyalkyl acrylates (e.g. hydroxyethyl acrylate), and trimethylolpropane. Preference is given to using monoalcohols such as methanol, ethanol and butanol.

6) Subject matter of the invention are also carbodiimides and/or uretonimines based on dianhydrohexitol-based diisocyanates, preferably of the formula I-III. The carbodiimidization of isocyanates is an operation which is known per se. Accordingly, processes for preparing isocyanate mixtures containing carbodiimide and/or uretonimine groups using the catalysts of the phospholene oxide series that are highly effective for this reaction, are known from U.S. Pat. No. 2,853,473 and EP 0 515 933 A, for example.

The carbodiimides and/or uretonimines of the invention are prepared in the presence of high-activity, phosphorus-containing catalysts, preferably of the phospholene oxide type.

An exhaustive description of suitable catalysts and preparation methods is found, for example, in Houben-Weyl, Methoden der organischen Chemie, volume XiV/1, Makromolekulare Stoffe [Macromolecular compounds], Georg-Thieme-Verlag, Stuttgart, 1984, pp. 897 to 910, and also in Chemical Reviews, volume 67, number 2, 1967, pp. 107-113, or in Angew. Chem., 1962, No. 21, 801-806. Carbodiimidization catalysts are also described in U.S. Pat. No. 2,941,966, U.S. Pat. No. 2,853,518, U.S. Pat. No. 2,853,473 or DE 3512918. Examples of catalysts employed with preference are 1-methyl-1-phospha-2-cyclopentene 1-oxide, 1-methyl-1-phospha-3-cyclopentene 1-oxide, 3-methyl-1-phenyl-3-phospholene 1-oxide and 3-methyl-1-phenyl-2-phospholene 1-oxide. According to safety data sheets from the manufacturers, e.g. Alfa Aesar, these phosphorus-containing catalysts are considered to pose a health hazard. Particular preference is given to using 3-methyl-1-phenyl-2-phospholene 1-oxide. The amount of catalyst relative to the diisocyanate is 0.1% to 3% by weight, preferably 0.5%-1.5% by weight.

The carbodiimides and/or uretonimines of the invention are preferably accessible by a process in which dianhydrohexitol-based diisocyanates are heated to temperatures of 30-200° C. with addition of the recited catalysts and with elimination of carbon dioxide, to prepare a polycarbodiimide mixture. The temperature is preferably 80-200° C., the duration between 30 minutes and 24 hours. Here, depending on catalyst content, temperature and time, there remain smaller to larger amounts of monomeric diisocyanates in the reaction mixture.

The derivatives of the invention may comprise further di- and polyisocyanates from any desired aromatic, aliphatic, cycloaliphatic and/or (cyclo)aliphatic di- and/or polyisocyanates.

Suitable aromatic di- or polyisocyanates are in principle all known compounds. Particularly suitable are 1,3- and 1,4-phenylene diisocyanate, 1,5-naphthylene diisocyanate, tolidine diisocyanate, 2,6-tolylene diisocyanate, 2,4-tolylene diisocyanate (2,4-TDI), 2,4′-diphenylmethane diisocyanate (2,4′-MDI), 4,4′-diphenylmethane diisocyanate, the mixtures of monomeric diphenylmethane diisocyanates (MDI) and oligomeric diphenylmethane diisocyanates (polymeric MDI), xylylene diisocyanate, tetramethylxylylene diisocyanate and triisocyanatotoluene.

Suitable aliphatic di- or polyisocyanates possess advantageously 3 to 16 carbon atoms, preferably 4 to 12 carbon atoms, in the linear or branched alkylene radical, and suitable cycloaliphatic or (cyclo)aliphatic diisocyanates advantageously possess 4 to 18 carbon atoms, preferably 6 to 15 carbon atoms, in the cycloalkylene radical. (Cyclo)aliphatic diisocyanates are understood sufficiently by the skilled person to involve NCO groups attached both cyclically and aliphatically, as is the case with isophorone diisocyanate, for example. In contrast, cycloaliphatic diisocyanates are understood to be those which have only NCO groups attached directly to the cycloaliphatic ring, an example being H₁₂MDI. Examples are cyclohexane diisocyanate, methylcyclohexane diisocyanate, ethylcyclohexane diisocyanate, propylcyclohexane diisocyanate, methyldiethylcyclohexane diisocyanate, propane diisocyanate, butane diisocyanate, pentane diisocyanate, hexane diisocyanate, heptane diisocyanate, octane diisocyanate, nonane diisocyanate, nonane triisocyanate, such as 4-isocyanatomethyl-1,8-octane diisocyanate (TIN), decane diisocyanate and triisocyanate, undecane diisocyanate and triisocyanate, dodecane diisocyanates and triisocyanates.

Preference is given to isophorone diisocyanate (IPDI), hexamethylene diisocyanate (HDI), diisocyanatodicyclohexylmethane (H₁₂MDI), 2-methylpentane diisocyanate (MPDI), 2,2,4-trimethylhexamethylene diisocyanate/2,4,4-trimethylhexamethylene diisocyanate (TMDI), norbornane diisocyanate (NBDI). Very particular preference is given to using IPDI, HDI, TMDI and H₁₂MDI, and the isocyanurates can be used as well.

Likewise suitable are 4-methylcyclohexane 1,3-diisocyanate, 2-butyl-2-ethylpentamethylene diisocyanate, 3(4)-isocyanatomethyl-1-methylcyclohexyl isocyanate, 2-isocyanatopropylcyclohexyl isocyanate, 2,4′-methylenebis(cyclohexyl) diisocyanate, and 1,4-diisocyanato-4-methylpentane.

It is of course also possible to use mixtures of the di- and polyisocyanates.

In addition use is made preferably of oligoisocyanates or polyisocyanates which are preparable from the stated di- or polyisocyanates or mixtures thereof by linking by means of urethane, allophanate, urea, biuret, uretdione, amide, isocyanurate, carbodiimide, uretonimine, oxadiazinetrione or iminooxadiazinedione structures. Particularly suitable are isocyanurates, especially those formed from IPDI and HDI.

A further subject of the present invention is the use of the derivatives of the invention as coating materials, more particularly as primer, tiecoat, topcoat, clearcoat, adhesive or sealing material, and also the coating materials themselves.

Subject matter of the invention is also the use of the derivatives of the invention for producing coatings in liquid and powder form on metal, plastics, glass, wood, textile, MDF (Middle Density Fiber Boards) or leather substrates, or other heat-resistant substrates.

Subject matter of the invention is also the use of the derivatives of the invention as adhesive compositions for adhesive bonds of metal, plastics, glass, wood, textile, MDF (Middle Density Fiber Boards) or leather substrates, or other heat-resistant substrates.

Likewise subject matter of the invention are metal-coating compositions, more particularly for automobile bodies, motor and pedal cycles, architectural components and household appliances, wood-coating compositions, glass-coating compositions, textile-coating compositions, leather-coating compositions and plastics-coating compositions which comprise the derivatives.

The coating may either be used alone or may be one coat in a multicoat system. It may be applied, for example, as a primer, as a tiecoat or as a topcoat or clearcoat. The coats situated above or below the coating may be cured either conventionally, thermally, or else, alternatively, by radiation.

### EXAMPLES

### 1) Dimers (Uretdione)

### a) Comparative

20 g of 2,5-diisocyanato-1,4:3,6-dianhydro-2,5-dideoxy-L-iditol (III) are introduced and 0.2 g of sodium 1,2,4-triazolate in solution in 2 ml of DMSO is added. After two minutes, the mixture begins to develop heat and to foam. Within a few minutes it has become solid. The resultant solid is no longer soluble and according to its IR spectrum (KBr) no longer contains any free isocyanate.

This reaction shows that isocyanates based on dianhydrohexitols, in contrast to conventional isocyanates, tend toward unusual reactions which cannot be simply predicted in every case.

### b) Inventive

20 g of 2,5-diisocyanato-1,4:3,6-dianhydro-2,5-dideoxy-L-iditol (III) are introduced and 0.2 g of 4-dimethylaminopyridine in solution in 2 ml of methylene chloride is added. After 5 days of stirring at room temperature the solution is freed from monomeric diisocyanates by bulb-tube distillation at 90° C. and 0.03 mbar. The latent NCO content (uretdione, by titrimetry) is 11%. The monomer content is 0.4% by weight. In the 13-C NMR, the position of the uretdione carbonyl C-atom can be seen at 156.5 ppm. In the IR, it is possible to make out the uretdione peak clearly at a wavenumber of 1780 cm⁻¹.

### 2) Trimers (Isocyanurates)

20 g of 2,5-diisocyanato-1,4:3,6-dianhydro-2,5-dideoxy-L-iditol (III) are introduced and 0.5 g of DABCO-TMR (trimerization catalyst, Air Products) is added. The mixture is then heated to 100° C. and cooled after 20 minutes. The resultant product is freed from monomeric diisocyanates by bulb-tube distillation at 90° C. and 0.03 mbar. The free NCO content is 19.3%; the monomer content is 0.2% by weight. In the 13-C NMR, the position of the isocyanurate carbonyl C-atom can be seen at 148.3 ppm. In the IR, it is possible to make out the isocyanurate peak clearly at a wavenumber of 1690 cm⁻¹.

### 3) NCO Prepolymers

17.6 g of 2,5-diisocyanato-1,4:3,6-dianhydro-2,5-dideoxy-L-iditol (III) are dissolved in 200 ml of acetone, this solution is mixed with 44.7 g of Oxyester T 1136 (polyester, neopentylglycol adipate, Evonik Degussa GmbH) (NCO/OH=2:1) and 0.03 g of dibutyltin dilaurate is added. After 6 hours at 60° C., the reaction product is cooled and the solvent is stripped off on a rotary evaporator. The product has a free NCO content of 6.1% and a monomer content of 2.9% by weight. Free OH groups cannot be detected.

### 4) Blocked Isocyanates

30 g of 2,5-diisocyanato-1,4:3,6-dianhydro-2,5-dideoxy-L-iditol (III) are dissolved with 36 g of ε-caprolactam in 100 ml of toluene and the solution is boiled under reflux for 1 hour. The solvent is thereafter removed on a rotary evaporator. The resultant product has an NCO content of <0.1% and a monomer content of <0.1% by weight.

### 5) Allophanates

60 g of 2,5-diisocyanato-1,4:3,6-dianhydro-2,5-dideoxy-L-iditol (III) are admixed with 11.3 g of butanol and 0.01 g of dibutyltin dilaurate and heated at 50° C. for 5 hours. Following complete reaction (NCO content 26.6%), 1 g of zinc octoate is added and the mixture is heated at 110° C. for 4 hours. The NCO content thereafter is 20.3%. The excess monomer is removed on a bulb-tube distillation apparatus. Thereafter the monomer content is 1.4% by weight and the NCO content is 13.4%.

### 6) Carbodiimides

20 g of 2,5-diisocyanato-1,4:3,6-dianhydro-2,5-dideoxy-L-iditol (III) are dissolved in 100 ml of toluene and admixed with 0.2 g of 3-methyl-1-phenyl-2-phospholene 1-oxide (Alfa Aesar). The mixture is then boiled under reflux for 24 hours. The resultant product has a carbodiimide content (as NCN) of 20.3% and a monomer content of 8.3% by weight. In the 13-C NMR, the position of the carbodiimide carbonyl C-atoms can be seen at 137-138 ppm.

1. A derivative of a dianhydrohexitol-based diisocyanate, comprising at least one selected from the group consisting of
1) a dimer (uretdione),
2) a trimer (isocyanurate),
3) an NCO-containing prepolymer having at least one free, blocked, or both, NCO group,
4) a blocked diisocyanate,
5) an allophanate,
6) a carbodiimide, a uretonimine, or both;
wherein:
the derivative possesses a free NCO group, a blocked NCO group, or both; and
a content of one or more monomeric diisocyanates is less than 20% by weight.

2. The derivative of claim 1, wherein the dianhydrohexitol-based diisocyanate is at least one selected from the group consisting of 2,5-diisocyanato-1,4:3,6-dianhydro-2,5-dideoxy-D-mannitol (I), 2,5-diisocyanato-1,4:3,6-dianhydro-2,5-dideoxy-D-glucitol (II) and 2,5-diisocyanato-1,4:3,6-dianhydro-2,5-dideoxy-L-iditol (III), corresponding to formulae 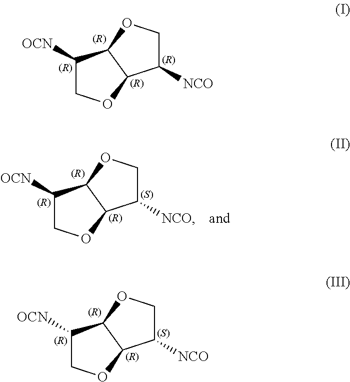
.

3. The derivative of claim 1, wherein
the dimer 1) has a free NCO content between 1%-42% by weight, a uretdione content between 1% and 42% by weight and a monomer content between 0.5% and 98% by weight, and
the monomer content after distillation is 0%-20% by weight.

4. The derivative of claim 1, wherein
the dimer 1) has reacted with at least one hydroxyl-containing monomer or polymer, as a chain extender and optionally with at least one monoamine, monoalcohol, or both, as a chain terminator and has a free NCO content of less than 5% by weight and a uretdione content of 2% to 25% by weight (calculated as C₂N₂O₂, molecular weight 84).

5. A process for preparing the dimer 1) of claim 1, the process comprising reacting the dianhydrohexitol-based diisocyanate at room temperature in the presence of a catalyst.

6. The derivative of claim 1 wherein
the trimer 2) has a free NCO content after reaction of 1%-42% by weight and a monomer content between 0.5% and 98% by weight.

7. The derivative of claim 6, wherein
the trimer 2) is blocked with at least one blocking agent selected from the group consisting of a phenol, an alcohol, an oxime, an N-hydroxy compound, a lactam, a CH-acidic compound, an amine, a heterocyclic compound having at least one heteroatom, an α-hydroxybenzoic ester and a hydroxamic ester.

8. The derivative of claim 7, wherein the blocking agent is at least one selected from the group consisting of acetone oxime, methyl ethyl ketoxime, acetophenone oxime, diisopropylamine, 3,5-dimethylpyrazole, 1,2,4-triazole, ε-caprolactam, butyl glycolate, benzyl methacylohydroxamate, methyl p-hydroxybenzoate.

9. A process for preparing the trimer 2) of claim 1, the process comprising
trimerization of the dianhydrohexitol-based diisocyanate in the presence of at least one catalyst optionally with at least one solvent, auxiliary, or both.

10. The process of claim 9,
wherein
the trimerization occurs with at least one quaternary hydroxyalkylammonium carboxylate at a temperature range of 40 to 140° C.

11. The derivative of claim 1, wherein
the NCO-containing prepolymer 3) is obtained by reaction of the dianhydrohexitol-based diisocyanate and one or more at least difunctional polyol in an NCO/OH ratio of 1.5-2:1 at 20 to 120° C.

12. The derivative of claim 11, wherein
the NCO-containing prepolymer 3) has a monomer content after reaction of between 0.5% to 20% by weight and a monomer content after distillation is less than 2% by weight.

13. The derivative of claim 11, further comprising at least one
diisocyanate selected from the group consisting of hexamethylene diisocyanate (HDI), isophorone diisocyanate (IPDI), 4,4′-methylenebis(cyclohexyl isocyanate) (H₁₂MDI), 2-methylpentane-methylene-1,5-diisocyanate (MPDI), trimethylhexamethylene-1,6-diisocyanate (TMDI), and m-tetramethylxylylene diisocyanate (TMXDI).

14. The derivative of claim 11, wherein the at least difunctional polyol is at least one selected from the group consisting of
ethylene glycol, 1,2-propanediol, 1,3-propanediol, diethylene glycol, dipropylene glycol, triethylene glycol, tetraethylene glycol, 1,2-butanediol, 1,4-butanediol, 1,3-butylethylpropanediol, 1,3-methylpropanediol, 1,5-pentanediol, bis(1,4-hydroxymethyl)cyclohexane (cyclohexanedimethanol), glycerol, hexanediol, neopentylglycol, trimethylolethane, trimethylolpropane, pentaerythritol, bisphenol A, bisphenol B, bisphenol C, bisphenol F, norbornylene glycol, 1,4-benzyldimethanol, 1,4-benzyldiethanol, 2,4-dimethyl-2-ethylhexane-1,3-diol, 1,4- and 2,3-butylene glycol, di-β-hydroxyethylbutanediol, 1,5-pentanediol, 1,6-hexanediol, 1,8-octanediol, decanediol, dodecanediol, neopentylglycol, cyclohexanediol, 3(4),8(9)-bis(hydroxymethyl)tricyclo-[5.2.1.02,6]decane (Dicidol), 2,2-bis(4-hydroxycyclohexyl)propane, 2,2-bis[4-(β-hydroxyethoxy)phenyl]propane, 2-methylpropane-1,3-diol, 2-methylpentane-1,5-diol, 2,2,4(2,4,4)-trimethylhexane-1,6-diol, hexane-1,2,6-triol, butane-1,2,4-triol, tris(β-hydroxyethyl)isocyanurate, mannitol, sorbitol, a polypropylene glycol, a polybutylene glycol, xylylene glycol hydroxypivalate and neopentylglycol hydroxypivalate.

15. The derivative of claim 11, wherein the at least difunctional polyol is at least one selected from the group consisting of a
linear or branched hydroxyl-containing polyester, polycarbonate, polycaprolactone, polyether, polythioether, polyesteramide, polyacrylate, polyurethane and polyacetal.

16. The derivative of claim 11, wherein
the NCO-containing prepolymer 3) is blocked with at least one blocking agent selected from the group consisting of acetone oxime, methyl ethyl ketoxime, acetophenone oxime, diisopropylamine, 3,5-dimethylpyrazole, 1,2,4-triazole, ε-caprolactam, butyl glycolate, benzyl methacylohydroxamate, and methyl p-hydroxybenzoate.

17. The derivative of claim 1, wherein the blocked diisocyanate 4) is blocked with at least one
blocking agent selected from the group consisting of a phenol, an alcohol, an oxime, an N-hydroxy compound, a CH-acidic compound, an amine, a heterocyclic compound having at least one heteroatom, an α-hydroxybenzoic ester and a hydroxamic ester.

18. The derivative of claim 17, wherein the
blocking agent is at least one selected from the group consisting of acetone oxime, methyl ethyl ketoxime, acetophenone oxime, diisopropylamine, 3,5-dimethylpyrazole, 1,2,4-triazole, ε-caprolactam, butyl glycolate, benzyl methacylohydroxamate, and methyl p-hydroxybenzoate.

19. The derivative of claim 17, wherein
a ratio between an NCO component and the blocking agent is 1:1 to 1:1.2 and the blocked diisocyanate 4) has an NCO content of less than 0.5% by weight.

20. A process for preparing the blocked diisocyanate 4) of claim 1, the process comprising
reacting a diisocyanate with at least one blocking agent at temperatures between room temperature and 220° C.

21. The derivative of claim 1, wherein
the allophanate 5) is prepared with at least one selected from the group consisting of methanol, ethanol, a propanol isomer, a butanol isomer, a pentanol isomer, a hexanol isomer, an octanol isomer, a decanol isomer, a dodecanol isomer, ethylene glycol, 1,2-propanediol, 1,3-propanediol, diethylene glycol, dipropylene glycol, triethylene glycol, tetraethylene glycol, 1,2-butanediol, 1,4-butanediol, 1,3-butylethylpropanediol, 1,3-methylpropanediol, 1,5-pentanediol, bis(1,4-hydroxymethyl)cyclohexane (cyclohexanedimethanol), glycerol, hexanediol, neopentylglycol, trimethylolethane, trimethylolpropane, pentaerythritol, bisphenol A, bisphenol B, bisphenol C, bisphenol F, norbornylene glycol, 1,4-benzyldimethanol, benzyldiethanol, 2,4-dimethyl-2-ethylhexane-1,3-diol, 1,4-butylene glycol and 2,3-butylene glycol, di-β-hydroxyethylbutanediol, 1,5-pentanediol, 1,6-hexanediol, 1,8-octanediol, decanediol, dodecanediol, neopentylglycol, cyclohexanediol, 3(4),8(9)-bis(hydroxymethyl)tricyclo-[5.2.1.0^{2,6}]decane (Dicidol), 2,2-bis(4-hydroxycyclohexyl)propane, 2,2-bis[4-(β-hydroxyethoxy)phenyl]propane, 2-methylpropane-1,3-diol, 2-methylpentane-1,5-diol, 2,2,4(2,4,4)-trimethylhexane-1,6-diol, hexane-1,2,6-triol, butane-1,2,4-triol, tris(β-hydroxyethyl)isocyanurate, mannitol, sorbitol, a polypropylene glycol, a polybutylene glycol, xylylene glycol hydroxypivalate, neopentylglycol hydroxypivalate, a hydroxyalkyl acrylate, and trimethylolpropane.

22. The derivative of claim 21, wherein
the allophanate 5) has a monomer content after distillation of less than 2% by weight.

23. A process for preparing the allophanate 5) of claim 1, the process comprising
addition of at least one alcohol in a substoichiometric amount to a dianhydrohexitol-based diisocyanate in a urethane reaction and, after complete reaction according to an NCO content analysis,
adding an allophanatization catalyst to affect an allophanatization reaction at 80 to 140° C. in 30 minutes to 8 hours, until change in the NCO content is no longer detected.

24. The derivative of claim 1, wherein
the carbodiimide, uretonimine, or both, 6) has NCO content, monomer content, or both.

25. A process for preparing the carbodiimide, uretonimine, or both, 6), the process comprising reacting the dianhydrohexitol-based diisocyanate in the presence of at least one high-activity, phosphorus-containing catalyst.

26. A coating material, comprising the derivative of claim 1.

27. An article comprising the coating material of claim 26, wherein the article is at least one selected from the group consisting of a primer, a tiecoat, a topcoat, a clearcoat, an adhesive material, and a sealing material, in a water-based, radiation-curable, powderous, solvent-free or solvent-containing system.

27. The use as claimed in claim 26 as primer, tiecoat, topcoat, clearcoat, adhesive or sealing material, in water-based, radiation-curable, powderous, solvent-free or solvent-containing systems.
